(19) United States
(12) Patent Application Publication
(10) Pub. No.: US 2014/0031417 A1
(43) Pub. Date: Jan. 30, 2014

(54) SKELETAL MUSCLE-SPECIFIC ENHANCER

(71) Applicants: Frédéric Relaix, Paris (FR); Keren Bismuth, Paris (FR)

(72) Inventors: Frédéric Relaix, Paris (FR); Keren Bismuth, Paris (FR)

(73) Assignee: UNIVERSITE PIERRE ET MARIE CURIE (PARIS 6), Paris (FR)

(21) Appl. No.: 14/009,971

(22) PCT Filed: Apr. 5, 2012
(86) PCT No.: PCT/EP2012/056361

§ 371 Date: Oct. 4, 2013

(30) Foreign Application Priority Data
Apr. 6, 2011 (EP) 11305402.7

(51) Int. Cl. C12N 15/85

(52) U.S. Cl. CPC C12N 15/85
USPC 514/44 R; 435/320.1

(57) ABSTRACT

The invention relates to a novel skeletal muscle-specific p57 regulatory element. This element is useful to drive and enhance the specific skeletal muscle expression of therapeutic genes. It is therefore provided nucleic acids and vectors comprising said enhancer element, as well as pharmaceutical compositions comprising the vectors, and uses thereof.

The invention provides a regulatory element that drives and enhances protein expression in skeletal muscle.

### BACKGROUND OF THE INVENTION

Gene therapy is the intracellular delivery of exogenous genetic material that corrects an existing defect or provides a new beneficial function to the cells. The muscle is an important target tissue for gene therapy because of its ready accessibility for direct injection, a relatively easy and minimally invasive method. Additionally, the muscle permits greater expression persistence compared to tissues with a higher cellular turnover rate. Skeletal muscle, for example, is being explored as a target tissue for gene therapy in a variety of therapeutic applications. There are a large number of known diseases caused by defects in gene products that could benefit from production of a protein secreted by the muscle. Familial hypercholesterolemia, hemophilia, Gaucher's and Fabry diseases, and type II diabetes are just a few examples. Many such diseases may be amenable to gene therapy. Muscle based diseases may especially benefit from gene therapy that would specifically target the skeletal muscle.

Muscle based diseases are manifested in a multitude of fashions, from hereditary, to acquired disorders. The classic example is muscular dystrophy, of which there are several major types, which can appear from infancy to adulthood, and which affect over 300,000 European citizens. Acquired muscular disorders can arise from infections, problems of auto-immunity, alcoholism, metabolic disorders or traumatic events.

Various expression vectors have been developed to deliver exogenous genetic material into various tissues and organs, and muscle tissue, in particular. Generally, each expression system possesses certain disadvantages and obtaining desired levels of expression in vivo in a sustainable manner can be a challenge. Various gene delivery vectors, such as adeno-associated virus (AAV), have been designed to achieve skeletal muscle gene transfer for the treatment of muscular dystrophies for instance. However, the use of ubiquitous viral promoters represents a major safety issue that could limit their use. By using strategies that avoid gene expression in specific cell types such as antigen presenting cells, it may be possible to abrogate unwanted immune responses against a desired gene.

There is thus a great need for efficient transcriptional regulatory systems that can direct high level of therapeutic genes in a cell type-specific manner. Such systems would be extremely useful in various gene therapy approaches, because most gene therapy procedures to date have used viral or other nonregulated promoters that have resulted in nonspecific expression in most cell types, even those in which expression was not necessarily desired.

### SUMMARY OF THE INVENTION

The present invention relates to the use of a compact and highly specific enhancer for gene therapy. This construct is useful in gene therapy approaches to target and enhance gene expression in a tissue specific manner.

More particularly inventors have identified a novel skeletal muscle-specific p57 regulatory element. Beyond the regulation of p57, this element can be taken out of its context and drive and enhance the specific skeletal muscle expression of other genes or cassettes.

More particularly the invention provides an isolated transcriptional Regulatory Element nucleic acid that consists of, or comprises (i) sequence SEQ ID NO:1, or (ii) a homologous sequence defined as showing at least 40% identity with SEQ ID NO:1 while retaining its biological activity as a transcriptional Regulatory Element that enhances protein expression in skeletal muscle specifically.

The invention further provides a vector, such as a virus or a plasmid, that comprises the Regulatory Element as defined herein, and a nucleic acid sequence of interest under the control of a promoter.

Another subject of the invention is a pharmaceutical composition comprising said vector in association with a pharmaceutically acceptable carrier.

The vector or pharmaceutical composition may be particularly useful for in vivo delivery to muscle.

Generally speaking, the vector or pharmaceutical composition may be for treating any disease that may benefit for gene therapy in the muscle, especially a musculoskeletal disease.

### DESCRIPTION OF THE FIGURES

FIG. 1 shows in vivo expression of p57MRE-TKnLacZ.

### DETAILED DESCRIPTION OF THE INVENTION

The invention provides a skeletal muscle-specific transcriptional regulatory sequence that is (i) capable of targeting skeletal muscle expression (ii) mediating a robust expression (iii) short enough to challenge the size-constrain of many vectors used for gene therapy.

In order to understand how p57 is regulated during muscle development and in adulthood, the inventors looked for specific sequences that could regulate p57 expression in muscle cells.

The regulatory element of the invention is located at about +59 kb downstream of p57. It contains several transcription factors binding site, notably it has an elevated density of E-boxes. E-boxes are palindromes “CANNTG” DNA sequences, which are recognized and bound by basic Helix Loop Helix (bHLH) transcription factors, among them MyoD that is a major bHLH transcription factor expressed during muscle development and during muscle injury in the adult.

The inventors named this sequence p57 MRE for Muscle Regulatory Element.

Definitions

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wley & Sons, Inc. (1994).

A “nucleic acid ” can be RNA or a DNA molecule, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

An “isolated” nucleic acid is a nucleic acid, which is substantially separated from other components which naturally accompany a native sequence, e.g., ribosomes, polymerases, and flanking genomic sequences from the originating species. The term embraces a nucleic acid sequence which has been removed from its naturally occurring environment, and includes recombinant or cloned DNA isolates and chemically synthesized analogs or analogs biologically synthesized by heterologous systems.

The terms “vector”, or “expression vector” refer to the vehicle by which DNA can be introduced into a host cell, resulting in expression of the introduced sequence. In one embodiment, vectors comprise a promoter and one or more control elements that are heterologous to the introduced DNA but are recognized and used by the host cell. In another embodiment, the sequence that is introduced into the vector retains its natural promoter that may be recognized and expressed by the host cell (Bormann et al., J. Bacteriol 1996; 178:1216-1218).

The nucleic acids in the vector may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene” means a DNA sequence that codes for or corresponds to a particular sequence of amino acids which comprise all or part of one or more proteins or enzymes.

A coding sequence is “under the control of” or “operatively associated with” expression control sequences in a cell when RNA polymerase transcribes the coding sequence into RNA, particularly mRNA, which is then translated into the protein encoded by the coding sequence.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that encodes an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”. A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host, e.g. antibiotic resistance, and one or more expression cassettes. Vector constructs may be produced using conventional molecular biology and recombinant DNA techniques within the skill of the art.

The term “homologous” may be used to indicate a variant nucleic acid sequence, including homologous sequences from various animal species, allelic variants, or modified sequences. Generally two DNA sequences are “homologous” when at least about 40%, preferably at least 50%, 60%, 70%, 80% or at least 90% or 95% of the nucleotides match over the defined length of the DNA sequences, as determined by sequence comparison algorithms, such as BLAST, FASTA, DNA Strider, etc. Sequences that are homologous can be identified by comparing the sequences using standard software available in sequence data banks, or in a Southern hybridization experiment under, for example, stringent conditions as defined for that particular system.

Preferably the homologous sequences are “substantially identical” i.e. the sequence of nucleotides is the same when aligned for maximum correspondence as described below. Optimal alignment of sequences for comparison may be conducted by the local homology algorithm of Smith and Waterman, Add. APL. Math. 2:482 (1981), by the homology alignment algorithm of Needle man and Wunsch, J. Mol. Biol. 48:443 (1970), by the search for similarity method of Pearson and Lipman, Proc. Natl. Acad. Sci. U.S.A. 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection. “Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ e.g., 40% formamide, with 5× or 6×SCC. High stringency hybridization conditions correspond to the highest Tₘ, e.g., 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51).

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

The term “subject” or “patient” as used herein includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The term “therapeutically effective amount” refers to an amount effective, at dosages and for periods of time necessary, to achieve the desired therapeutic result. A therapeutically effective amount of the recombinant vector may vary according to factors such as the disease state, age, sex, and weight of the individual and the ability of the vector to elicit a desired response in the individual. A therapeutically effective amount is also one in which any toxic or detrimental effect is outweighed by the therapeutically beneficial effects.

The Enhancer Elements of the Invention:

The annexed sequence listing shows the minimal sequence of the human enhancer element of the invention (SEQ ID NO:1). Sequences SEQ ID NO: 2, 3, 4, 5, 6, 7, 8 are the corresponding sequences respectively in mouse, rat, gorilla, orangutan, macaque, dog, and horse.

Sequence SEQ ID NO: 9 is an extended sequence of the human enhancer element of the invention (SEQ ID NO:9). Sequences SEQ ID NO: 10, 11, 12, 13, 14, 15, 16 are the corresponding sequences respectively in mouse, rat, gorilla, orangutan, macaque, dog, and horse.

The invention provides an isolated transcriptional Regulatory Element nucleic acid that consists of, or comprises (i) sequence SEQ ID NO:1 or 9, or (ii) a homologous sequence defined as showing at least 40% identity said sequence while retaining its biological activity as a transcriptional RE that enhances protein expression in skeletal muscle specifically.

Preferably the Regulatory Element nucleic acid sequence is less than about 800 nucleotides.

The enhancer of the invention is thus designated as being “muscle-specific”, in that it is capable to drive or enhance expression of transgenes exclusively or preferentially in muscle tissue or muscle cells.

The invention provides an isolated transcriptional Regulatory Element nucleic acid that comprises a portion of SEQ ID NO:9, or a homologous sequence thereof, said portion comprising at least SEQ ID NO:1, or a homologous sequence thereof. In a preferred embodiment, said portion comprising between about 442 and about 730 bp, preferably between about 442 and about 500 bp.

The invention also provides an isolated transcriptional Regulatory Element nucleic acid that consists of, or comprises (i) sequence SEQ ID NO:2 or 10, or (ii) a homologous sequence defined as showing at least 40% identity said sequence while retaining its biological activity as a transcriptional RE that enhances protein expression in skeletal muscle specifically.

The invention further provides an isolated transcriptional Regulatory Element nucleic acid that comprises a portion of SEQ ID NO:10, or a homologous sequence thereof, said portion comprising at least SEQ ID NO:2, or a homologous sequence thereof. In a preferred embodiment, said portion comprising between about 427 and about 685 bp, preferably between about 427 and about 500 bp.

The homologous sequence is defined as showing at least 50%, preferably at least 60%, still preferably at least 70%, still preferably at least 80% or at least 90% or 95% identity with the reference sequence.

When the reference sequence is SEQ ID NO:1, examples of homologous sequences are sequences that comprise SEQ ID NO:2, 3, 4, 5, 6, 7 or 8.

When the reference sequence is SEQ ID NO: 9, examples of homologous sequences are sequences that comprise SEQ ID NO:10, 11, 12, 13, 14, 15 or 16.

Homologous sequences comprise sequences that hybridize to any of SEQ ID NO:1 to 16, especially any of SEQ ID NO: 1; 2, 9 or 10, or their complementary strand, under moderate or high stringency hybridization conditions, as defined above.

The homologous sequence differs from SEQ ID NO:1 to 16 by a substitution, a deletion or an insertion of one or several nucleotides.

In a preferred embodiment, the homologous sequence comprises at least 5, preferably at least 6, 7, 8, or 9, E-boxes of sequence CANNTG (SEQ ID NO:17). Still preferably, the homologous sequence differs from SEQ ID NO:1 to 16 by a substitution, deletion or insertion of at least one nucleotide, it being understood that at least 5, preferably at least 6, 7, 8, or 9, E-boxes of sequence CANNTG (SEQ ID NO:17).

Vectors of the Invention:

The Regulatory Element of the invention can be inserted in a vector, especially an expression vector, comprising a nucleic acid sequence of interest under the control of a promoter.

Preferred vectors are adapted for gene therapy.

The promoter can be any minimal promoter, such as the Thymidine Kinase (TK) promoter, or a stronger promoter. Although ubiquitous promoters may be used, such as the CMV promoter, muscle cell specific promoters may be advantageous. Nonlimiting examples are muscle-specific creatine kinase promoter, desmin promoter, or myosin light chain promoters.

Other muscle-specific regulatory elements may be inserted. See e.g. US2003/0100526.

In the vector the regulatory element may be oriented 5′ to 3′ or 3′ to 5′. Indeed enhancer elements often remain active even if their orientation is reversed (Li et al., J. Bio. Chem. 1990, 266: 6562-6570). Furthermore, unlike promoter elements, enhancers can be active when placed downstream from the transcription initiation site, or even at a considerable distance from the promoter (Yutzey et al., Mol. and Cell. Bio. 1989, 9:1397-1405). The p57MRE of the invention can thus be placed upstream or downstream the nucleic acid sequence of interest, i.e. the transgene to express, and/or the promoter operably associated with said transgene.

As is known in the art, some variation in this distance can be accommodated without loss of promoter function. Similarly, the positioning of the regulatory element with respect to the transgene may vary significantly without loss of function. Multiple copies of regulatory elements can act in concert. Typically, an expression vector comprises one or more enhancer sequences followed by, in the 5′ to 3′ direction, a promoter sequence, all operably linked to a transgene followed by a polyadenylation sequence.

In general, there are no known limitations on the use of the regulatory elements of the invention in any vector. In some embodiments, the regulatory elements are incorporated in non-viral plasmid-based vectors. In other exemplary embodiments, a regulatory element of the invention is incorporated into a viral vector such as derived from adenoviruses, adeno-associated viruses (AAV), or retroviruses, including lentivirus.

The nucleic acid sequence of interest, also designated as “the transgene” may be a “therapeutic gene”, i.e. a sequence that encodes a therapeutic protein. The term “therapeutic gene” as used herein refers to a gene that, when expressed, confers a beneficial effect on the cell or tissue in which it is present, or on a patient in which the gene is expressed. Examples of beneficial effects include amelioration of a sign or symptom of a condition or disease, prevention or inhibition of a condition or disease, or conferral of a desired characteristic. Therapeutic genes include genes that partially or wholly correct a genetic deficiency in the patient.

The “therapeutic gene” may comprise a DNA sequence encoding proteins involved in metabolic diseases, or disorders and diseases of muscle system, muscle wasting, or muscle repair. Vectors of the invention may include a transgene containing a sequence coding for a therapeutic polypeptide. For gene therapy, such a transgene is selected based upon a desired therapeutic outcome. It may encode, for example, antibodies, hormones, enzymes, receptors, or other proteins of interest or their fragments, such as, for example, TGF-beta receptor, glucagon-like peptide 1, dystrophin, leptin, insulin, pre-proinsulin, follistatin, PTH, FSH, IGF, EGF, TGF-beta, bone morphogeneteic proteins, other tissue growth and regulatory factors, growth hormones, and blood coagulation factors. For example, in treatment of a muscle disorder or disease, the transgene or nucleic acid sequence of interest may encode a therapeutic protein, such as dystrophin; LARGE; dysferlin; calpain 3; Dux 4; LAMA2; α-, β-, γ-, and δ-sarcoglycan; FKRP, fukutin, WASP, factor VIII, factor IX, SMN1, U7, modified U7 (WO2006/021724), U1, RdCVF, α-glucosidase, meganuclease or zinc-finger nuclease, and actin. In principle, however, any gene that encodes a protein or polypeptide that is either reduced or absent due to a mutation or which conveys a therapeutic benefit when overexpressed is considered to be within the scope of the invention.

Pharmaceutical Compositions:

For in vivo delivery to cells, tissues, or organs of a subject, the vectors of the invention can be incorporated into pharmaceutical compositions suitable for administration to a subject. Typically, the pharmaceutical composition comprises the vectors and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. Pharmaceutical compositions comprising the vector can be delivered as, for example, ageratum, sprays, oral suspensions, suppositories, eye drops, and injectable suspensions.

Pharmaceutical compositions for therapeutic purposes typically must be sterile and stable under the conditions of manufacture and storage. The composition can be formulated as a solution, microemulsion, dispersion, liposomes, or other ordered structure suitable to high vector concentration. Sterile injectable solutions can be prepared by incorporating the vector compound in the required amount in an appropriate buffer with one or a combination of ingredients, as required, followed by filtered sterilization.

For in vivo administration of vectors, broad distribution of the vectors can be achieved in muscle by, for example, intravenous, intra-arterial injection or hydrodynamic locoregional perfusion. Depending on dosage and mode of delivery, 20-95% of muscle fibres in a given target muscle can be transduced. Particularly preferred delivery methods are those that deliver the vector to specific regions of the muscle that require expression of a polypeptide, encoded by the vector. Direct injection of vectors, whether subcutaneous, intracranial, intramuscular, or intradermal, can take place using standard needle and syringe methodologies, or by needle-free technologies.

Appropriate doses will depend on the disease or subject being treated. When administered to a subject, dose will depend on the particular mammal being treated (e.g., human or nonhuman primate or other mammal), age and general condition of the subject to be treated, the severity of the condition being treated, the particular therapeutic protein in question, its mode of administration, among other factors. An appropriate effective amount can be readily determined by one of skill in the art.

A “therapeutically effective dose” will fall in a relatively broad range that can be determined through clinical trials and will depend on the particular application (neural cells will require very small amounts, while systemic injection would require large amounts). For example, for direct in vivo injection into skeletal muscle of a human subject, a therapeutically effective dose will be on the order of from about 1 μg to 100 g of the vector. If exosomes or microparticles are used to deliver the vector, then a therapeutically effective dose can be determined experimentally, but is expected to deliver from 1 μg to about 100 g of vector.

Medical Uses of the Vectors:

In another aspect, the invention allows for the in vivo expression of a polypeptide encoded by a transgene in cells in a subject such that therapeutic levels of the polypeptide are expressed. Thus in one embodiment, the invention relates to a method of delivering a selected gene into skeletal muscle cell or tissue.

It is thus described a method of treating a disease in a subject comprising introducing into a muscle cell or tissue of the subject a therapeutically effective amount of a vector of the invention, optionally with a pharmaceutically acceptable carrier. While the vector can be introduced in the presence of a carrier, such a carrier is not required.

Generally speaking there are a large number of known diseases caused by defects in gene products that could benefit from production of a protein secreted by the muscle. Familial hypercholesterolemia, hemophilia, Gaucher's and Fabry diseases, and type II diabetes are just a few examples.

Preferably genetic muscle disorders are targeted. The term “genetic muscle disorder” as used herein refers to, but is not limited to, a neuromuscular or musculoskeletal disease or disorder, including without limitation, a neuromuscular disease caused by dominant mutation(s), recessive mutation(s), X-linked nuclear DNA mutation(s), or mitochondrial DNA mutation(s); also including large-range chromatin deletions which may or may not contain a gene but impacts gene editing. Examples include dominant or recessive limb girdle muscular dystrophies, Duchenne and Becker muscular dystrophy, Myotonic Dystrophy, Facioscapulohumeral dystrophy, and others.

Any musculoskeletal disease, including muscle atrophy or dystrophy may benefit from administration of the vector of the invention.

In a preferred embodiment, the musculoskeletal disease can be a muscular dystrophy syndrome, such as fascioscapulohumeral, Emery-Deifuss, oculopharyngeal, scapulohumeral, a congenital muscular dystrophy, or hereditary distal myopathy.

Ex vivo delivery of the vector, especially to muscle cells, can be performed before grafting or administering the transformed muscle cells into the subject to treat. The muscle cells may be autologous to the subject to treat. They may be progenitor cells or differentiated cells.

In a particular embodiment it is further described a method of treating a genetic or acquired muscle disease or disorder, such as a musculoskeletal disease, in a subject by (1) establishing a myoblast culture from a muscle biopsy of a subject with the disease, (2) delivering a vector to myoblasts or to muscle tissue, the vector comprising the desired exogenous DNA sequence operably linked to control elements capable of directing transcription of the desired polypeptide encoded by the transgene, (3) collecting exosomes or microparticles harboring the vector bearing the transgene from the culture or collecting the vectors in the form of DNA, and (4) delivering the collected filled exosomes or microparticles or vectors in the form of DNA into the subject.

The p57MRE of the invention is sufficient to trigger robust skeletal muscle-specific expression of a transgene of interest. Its short size (less than about 800 bp) makes it possible to use in any vector for gene therapy.

The Examples below illustrate the invention without limiting its scope.

### EXAMPLES

Materials and Methods

Plasmid Construct for Transgenesis

The mouse p57 muscle regulatory element (p57MRE) (chr7, 150587238-150587924 bp) was synthesized by PCR; for cloning convenience Eag1 restriction sites were added to the forward and reverse primers used for amplification: forward 5′-AAGCGGCCGCACCCAGTTTGCCCAGTGTAG-3′ (SEQ ID NO: 18) and reverse 5′-AACGGCCGCCAGGTAAAGACACCCCAGA-3′ (SEQ ID NO:19). After Eag1 digestion, the 686 bp fragment (SEQ ID NO: 10) was cloned, respecting its genomic orientation, into the NotI site of pTKnLacZ(−) plasmid (Hadchouel et al., 2000, Development 127(20):4455-67). The fragment p57MRE-TKnLacZ was released by SacII/XhoI digestion and gel purified using Nucleobond plasmid purification kit (Macherey-Nagel) before injection into pronuclei.

Tissue Preparation, Immunohistochemistry, X-Gal Staining

Embryos and forelimbs were harvested, fixed 20 min and overnight, respectively, in PBS 4% paraformaldehyde at 4° C., then treated in PBS 15% sucrose for overnight at 4° C. and embedded in OCT. Frozen sections were permeabilized in PBS 0,1% Triton-X-100, blocked in PBS 3% Bovine Serum Albumin for 30 min at RT, then immunolabeled with the following primary antibodies for overnight at 4° C.: mouse anti-MyoD 5.8A (DAKO) rabbit anti-Myf5 C20 (Santa Cruz) rabbit anti-p57 H91 (Santa Cruz), mouse anti-β-galactosidase GAL 13 (Sigma), rabbit anti β-galactosidase (Molecular probe). Secondary antibodies were coupled to: Alexa 488, 546 (Molecular probe) or Cy5 (Jackson ImmunoResearch). For X-Gal staining, embryos were collected in PBS, fixed 20 min in PBS 4% paraformaldehyde at RT, and incubated in X-gal solution (Invitrogen) overnight at 37° C. on a rotary shaker.

Chromatin Immuno-Precipitation, ChIP-Sequencing

MyoD ChIP-sequencing was described in detail before (Cao et al., 2010, Dev Cell 18(4): 662-674). For regular ChIP, forelimbs from E12,5 embryos were frozen in liquid nitrogen and processed for ChIP according to the manufacturer protocol (Active motif). Briefly, after fixation in 1% formaldehyde for 15 min, cells were lyzed and nuclei were sonicated for 17 pulses of 20 sec to obtain DNA fragments from 200 to 1000 bp. 150 μg of chromatin was used for each experiment. Rabbit anti-MyoD M318 (Santa Cruz), and as negative control rabbit anti-β-galactosidase (Molecular Probe) were used. The precipitated and input chromatins were next analyzed by quantitative PCR.

Reverse Transcription and Quantitative PCR

Total RNA from embryo forelimbs or FACS-sorted cells were extracted using RNAeasy mini kit (Qiagen). 1 μg of such RNA was used to generate cDNA using Superscript II reverse transcriptase kit (Invitrogen). Quantitative PCR were performed using lightcycler 480 sybergreen mix (Roche) and a Light cycler 480 II (Roche).

Transfection Assay

C2C12 adult muscle cell line cultured in DMEM 10% FBS, Penicilline/Streptomycine, were transfected with 1 μg of pTKnLacZ(−) or 1 μg p57MRE-TKnLacZ. The next day, C2C12 cells were cultured in differentiated medium (DMEM 5% FBS). After 3 days of differentiation in vitro, cells were fixed and assayed for the presence of LacZ.

### Example 1

### p57MRE is Identified

The inventors have investigated how cell cycle exit is regulated during myogenesis. They have demonstrated that both determined and differentiated myoblasts express p57, an effector cell cycle exit; p57 is a Cyclin Dependent Kinase Inhibitor (CDKI) and belongs to the Cip/Kip family of CDKI. They have also demonstrated that determined myoblasts, via Notch signaling pathway, prevent the activation of p57 in progenitor cells. To get some insights into the molecular details of p57 regulation, the inventors have then searched putative MyoD binding sites within the p57 locus (Cao et al, 2010, Dev Cell 18(4): 662-674). MyoD is a bHLH transcription factor and a major myogenic transcription factor.

The screen proposed by Cao et al. allowed the systematic identification of MyoD binding sites in the entire genome. The inventors hypothesized that p57 could be a target of MyoD, if this were the case then MyoD should be found at the p57 locus in the above screen. This screen was made using differentiated C2C12 cells, a well-characterized adult muscle cell line. A high density of MyoD binding sites was found at +59 Kb of murine p57.

The inventors isolated a conserved 686 by fragment that contains fifteen E-boxes. The inventors then performed ChIP experiments on E12,5 wild-type forelimbs, and found that MyoD was bound in vivo to this fragment.

### Example 2

### Functionality of the p57MRE is Tested

Next, the inventors assayed the functionality of the p57MRE sequence. They amplified and cloned the murine p57MRE in the Not1 site of pTKnLacZ(−) vector. After transfection in C2C12 cells, they observed a spontaneous expression of p57 MRE-TKnLacZ in 3 days differentiated myocytes and myotubes. The inventors attribute the LacZ reactivity to the presence of endogenous Myogenic Factors, which can directly activate the p57MRE.

### Example 3

### A Skeletal Muscle-Specific Expression Cassette is Provided

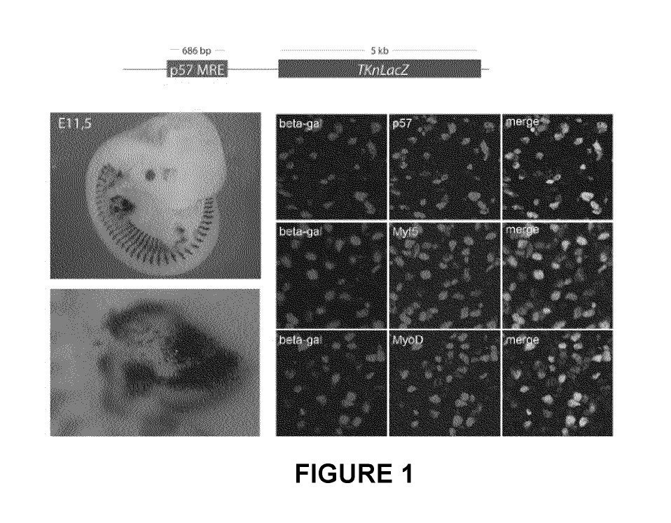

Finally, the inventors tested if this sequence could indeed enhance a skeletal muscle expression in vivo. They made transient transgenic experiments and observed a robust expression of the LacZ in skeletal muscle domains only and while p57 is also express in the heart, they never observed such expression with the p57MRE-TKnLacZ construct. More specifically the inventors indeed found that a p57MRE-TK-nLacZ construct was sufficient to trigger robust LacZ expression in all p57 muscle domains (FIG. 1) in E11,5 embryos.

The inventors have further checked the beta-gal expression profile, and found that 100% of beta-gal cells were p57+ but also MyoD+ positive; hence using this element they obtained expression of the reporter cassette in skeletal muscle cells.

1. A vector that comprises a Regulatory Element, and a nucleic acid sequence of interest under the control of a promoter, wherein the Regulatory Element is a transcriptional Regulatory Element nucleic acid that consists of, or comprises (i) sequence SEQ ID NO:1, or (ii) a homologous sequence defined as showing at least 40% identity with SEQ ID NO:1 while retaining its biological activity as a transcriptional Regulatory Element that enhances protein expression in skeletal muscle specifically.

2. The vector of claim 1, wherein the homologous sequence is defined as showing at least 50%, preferably at least 60%, still preferably at least 70%, still preferably at least 80% or at least 90% identity with SEQ ID NO:1.

3. The vector of claim 1 wherein the homologous sequence comprises SEQ ID NO:2, 3, 4, 5, 6, 7 or 8.

4. The vector of claim 1, wherein the Regulatory Element consists or comprises (i) nucleic acid sequence SEQ ID NO:9, or (ii) a homologous sequence defined as showing at least 40% identity with SEQ ID NO:9 while retaining its biological activity as a transcriptional Regulatory Element that enhances protein expression in skeletal muscle specifically.

5. The vector of claim 4, wherein the homologous sequence is defined as showing at least 50%, preferably at least 60%, still preferably at least 70%, still preferably at least 80% or at least 90% identity with SEQ ID NO:9.

6. The vector of claims 4, wherein the homologous sequence comprises SEQ ID NO:10, 11, 12, 13, 14, 15 or 16.

7. The vector of claim 1 wherein the homologous sequence comprises at least 5, preferably at least 6, 7, 8, or 9, E-boxes of sequence CANNTG (SEQ ID NO:17).

8. The vector of claim 1 wherein the homologous sequence differs from SEQ ID NO:1 to 16 by a substitution, deletion or insertion of at least one nucleotide, it being understood that at least 5, preferably at least 6, 7, 8, or 9, E-boxes of sequence CANNTG (SEQ ID NO:17)

9. The vector of claim 1 wherein the nucleic acid sequence of interest encodes a therapeutic protein.

10. The vector of claim 1 that is a virus.

11. The vector of claim 1 wherein the Regulatory Element is oriented 5′→33′ or 3′→5′.

12. A pharmaceutical composition comprising a vector as defined in claim 1 in association with a pharmaceutically acceptable carrier.

13. The vector or composition of claim 1, for in vivo delivery to muscle.

14. The vector or composition of claim 1 for use in treating a musculoskeletal disease.


SEQUENCE LISTING (19 sequences)

SEQ ID NO: 1 - DNA, 450 bp, Homo sapiens
catgagccagatgatgggagctgtcacaggactttgccacccggggtgaggggcctagaacccctctccaccagatgccttacaccccccatccccacacgcacacagcttggaggctggaagcccccggaatgcggcccaccctgtctccagttgttccctaccagcccagctgctggtccctaaccgggcccacctgttgggtccttagcaccagctgcccagagcccccagcccctaccctagtctgatgacccaggcctttccccctgcccaggctagccttgtgctcagctgccagcacagcccccaccacacactccccaacaggtgtctccacaactgccaggagacagctcagcttagctgctcttcccccaacccccaggcccagcagaatcacatctaggagagtgggccacatgcctcttggaaggcaggtggcacaag

SEQ ID NO: 2 - DNA, 427 bp, Mus musculus
caccatgtgcctgggccacttggcttggctacccaagcaagggcccaagagagtgccccctttgtcccttcttcacaaatgctgtgtatctactccacacatgtgcacacagctcagaggctcttgcctttggaatgcagtccaccccacctccagttttcccctaagcagttcagctgctaagccctgaactctcccacctgccgggtccttagcaccagctgcccagagctcctggacccaggcacagctggatgaccacccctccgagctggctaggcctgctgtcagctgccagtaaagccctccatccttcccaacaggtgtccccacaactgctgggggggttgcttagctaggcagctattccactgacccaggcctgcagaatgatgtcacacttaccactgtggaaggaggtagcccg

SEQ ID NO: 3 - DNA, 427 bp, Rattus norvegicus
caccatgtgcctgggccacttggcttggctacccaagcaaggggccaagagagtgccccctttgtctcttcttcacaaatgccatgtatccactccacacatgtgcacacagctcagaggctcttgcctttggaatgcagtccacactacctccagttctcccctaagcaattcagctgctaagccctgaactctcccacctgtcgggtccttaacaccagctgcccagagctcctggatccagacacagctggatgaccacccctctgagctggctaggcctgttgtcagctgccagtaaagccctccatccttcccaacaggtgtccccacaactgctgggggattgcttagctaggcagctattcctctgtgccaggcctgcaggatgatgttggaggggtaggccacttaccactttggaaga

SEQ ID NO: 4 - DNA, 450 bp, Gorilla gorilla
catgagccagatgatgggagctgtcacaggactttgccacccggggtgaggggcctagaaacccctctccaccagatgccttacaccccccatccccacacgcacacagcttggaggctggaagcccccggaatgcggcccaccctgtctccagttgttccctaccagcccagctgctggtgcctaaccccgcccacctgttgggtccttagcaccagctgcccagagcccccagcccctaccctagtctgatgacccaggcctttccccctgccccggctagccttgtgctcagctgccagcacagcccccaccacacactccccaacaggtgtctccacaactgccaggagacagctcagcttagctgctcttcccccaacccccaggcccagcagaatcacatctaggagagtgggccacatgcctcttggaaggcaggtggcacaa

SEQ ID NO: 5 - DNA, 450 bp, Pongo pygmaeus
catgagccagatgatgggagctgtcacaggactttgccaccgggggtgaggggcctagaaacccctctccaccagatgccttacaccccccatccccacacgcacacagcttggaggctagaagcccccggaatgcggcccaccctgtctccagttgttccctaccagcccagctgctggtccctaaccccacccacctgttgggtccttagcaacagctgcccagagcccccagcccctaccctagtctgatgacccaggcctttccccctgcccaggctagccttgtgctcagctgccagcacagcccccaccacacactccccaacaggtgtctccacaactgccaggagacagctcagcttagctgctcttcccccaacccccaggcccagcagaatcacgtctaggagagtgggccacatgcctcttggaaggcaggtggcacaa

SEQ ID NO: 6 - DNA, 390 bp, Macaca mulatta
tctccaccagatgccttacatcccccacccccccacacacacagcttggagactagaagcccctggaatgcggcccaccctctctccagttgttccccaccagcccagctgctggtccctaaccccacccacctgttgggtccttagcaccagctgcccagagcccccaacccctaccctagtctgatgacccaggccctttcccctgcccagactagccctgtgctcagctgccagcacagcccccaccacacactctccaacaggtgtctccacaactgccaggagacagctcagcttagctgctctttcctctacccccaggcccagcagaatcacatctaggagagtgggccacatgcctcttggaaggcaagtggcacatgtggt

SEQ ID NO: 7 - DNA, 451 bp, Canis familiaris
ccactgtgcagaagggcccagagggacccccattgtgccctctcctgcccaaggcccccgtgtccacccccacatacacacacacacacacacacacacacacacacacacacacacagctcaggcactgtgacgtggccccaggtggcccactctgactccagttgttaccagtccagctgccaagcccctacggcccatctgtcaggccccaggtccccagtgtcagctgcccaaagtcctcctccccaggtccccccaagcctgcaggccctgctctcagctgccaccagggcccccaccacaccctcccccacaggcgtccgtcccgctgtccccaagccagcagagttgtgttgggggaggggggcagaccacttgtcacttcagaaggcaggtgacatgaggggtccagccaccagcagagtcacgcagcccctgcactagccct

SEQ ID NO: 8 - DNA, 451 bp, Equus caballus
aggacggtgccactgggggtgaggggtccagcaagtggccccattgtcctttcttttacaccagatcccctctgcccgcccccctcgcccccccgacacacacacacacagcttaggggtcggatctgccttctgggatgcagcccaccctgccgccagttgttgccagcccagctgctgagccccccaccccggcccacctgtcaggcccgcgggtcctcagcgccagctgcccagagcccccagccccagtctgatgacccaggcccttcccccggccaggccccgctctcagctgccagcacagcccccaccacaccctccccaacaggtgtccccacaactgcagggagatgccccggcagaattgtgccgggggggggggggggggggggctggggaggggacccctgtccccttcggaagggaggtgggaagaggggtccggccc

SEQ ID NO: 9 - DNA, 733 bp, Homo sapiens
  misc_feature (12)..(17): E box
agccaccgtcccaggtgtggctaaggcccccacagagtgtgcatcctaaggtggttcagaggtcctcagaaagtgccaagaggccttcctcagaggaagagcaagggtaggtgcctctggcatgagccagatgatgggagctgtcacaggactttgccacccggggtgaggggcctagaacccctctccaccagatgccttacaccccccatccccacacgcacacagcttggaggctggaagcccccggaatgcggcccaccctgtctccagttgttccctaccagcccagctgctggtccctaaccgggcccacctgttgggtccttagcaccagctgcccagagcccccagcccctaccctagtctgatgacccaggcctttccccctgcccaggctagccttgtgctcagctgccagcacagcccccaccacacactccccaacaggtgtctccacaactgccaggagacagctcagcttagctgctcttcccccaacccccaggcccagcagaatcacatctaggagagtgggccacatgcctcttggaaggcaggtggcacaagaggttcatcccaccctccagcattacccagccccacaggacccacatcctgctcccagctccagcctgaaaggagctagtcctctccagcccctttgaaaagaccagctggacattctccagggagattctgccctacaggattagggagtaccctcacctgg

SEQ ID NO: 10 - DNA, 686 bp, Mus musculus
  misc_feature (94)..(99): E box
acccagtttgcccagtgtagagtgcccaaaagccctcagaaaagacaaagaaagagacctttcagactagaaaacagaagcaggatactttgacatatgtcaccatgtgcctgggccacttggcttggctacccaagcaagggcccaagagagtgccccctttgtcccttcttcacaaatgctgtgtatctactccacacatgtgcacacagctcagaggctcttgcctttggaatgcagtccaccccacctccagttttcccctaagcagttcagctgctaagccctgaactctcccacctgccgggtccttagcaccagctgcccagagctcctggacccaggcacagctggatgaccacccctccgagctggctaggcctgctgtcagctgccagtaaagccctccatccttcccaacaggtgtccccacaactgctgggggggttgcttagctaggcagctattccactgacccaggcctgcagaatgatgtcacacttaccactgtggaaggaggtagcccgagggatctgacagactccacggaagttattggttctagagaaacggcacaccagtcatactcagctccagctgggttgggtcagacctatccttttaaagaccaactggacattggccagagaagtttggccctgaagggtctggggtgtctttacctg

SEQ ID NO: 11 - DNA, 699 bp, Rattus norvegicus
  misc_feature (91)..(96): E box
acccagtttgcccagtgtagagtgcccaaaagccctcagaaatgacaaagaaaggcttttcagactagaaaacagaagaggatactttgacatatgtcaccatgtgcctgggccacttggcttggctacccaagcaaggggccaagagagtgccccctttgtctcttcttcacaaatgccatgtatccactccacacatgtgcacacagctcagaggctcttgcctttggaatgcagtccacactacctccagttctcccctaagcaattcagctgctaagccctgaactctcccacctgtcgggtccttaacaccagctgcccagagctcctggatccagacacagctggatgaccacccctctgagctggctaggcctgttgtcagctgccagtaaagccctccatccttcccaacaggtgtccccacaactgctgggggattgcttagctaggcagctattcctctgtgccaggcctgcaggatgatgttggaggggtaggccacttaccactttggaagaaggtaacatgagggatccaatggaccccacagaagttattggcactagaggacccccacaccagtcatattcagctaaagctgggttgggtcagatcttcctgcccttttcaaagaccaactggatattggccagagaagttcagccctgaagtgtctgggctatctttacctgg

SEQ ID NO: 12 - DNA, 734 bp, Gorilla gorilla
  misc_feature (12)..(17): E box
agccaccgtcccaggtgtggctaaggcccccacagggtgtgcatcctaaggtggttcagaggtcctcagaaagcgccaagaggccttcctcagaggaagagcaagggtaggtgcctctggcatgagccagatgatgggagctgtcacaggactttgccacccggggtgaggggcctagaaacccctctccaccagatgccttacaccccccatccccacacgcacacagcttggaggctggaagcccccggaatgcggcccaccctgtctccagttgttccctaccagcccagctgctggtgcctaaccccgcccacctgttgggtccttagcaccagctgcccagagcccccagcccctaccctagtctgatgacccaggcctttccccctgccccggctagccttgtgctcagctgccagcacagcccccaccacacactccccaacaggtgtctccacaactgccaggagacagctcagcttagctgctcttcccccaacccccaggcccagcagaatcacatctaggagagtgggccacatgcctcttggaaggcaggtggcacaagaggttcatcccaccctccagcattacccagccccacaggacccacatcctgctcccagctccagcctgaaaggagctagtcctctccagcccctttgaaaagaccagctggacattctccagggagattctgccctacaggattagggagtaccctcacctgg

SEQ ID NO: 13 - DNA, 734 bp, Pongo pygmaeus
  misc_feature (12)..(17): E box
agccactgtcccaggtgtggctaaggcccccacagagtgtgcatcctaaggtggctcagagatcctcagaaagcgccaagaggccttcctcagaggaagagcaaggataggtgcctctggcatgagccagatgatgggagctgtcacaggactttgccaccgggggtgaggggcctagaaacccctctccaccagatgccttacaccccccatccccacacgcacacagcttggaggctagaagcccccggaatgcggcccaccctgtctccagttgttccctaccagcccagctgctggtccctaaccccacccacctgttgggtccttagcaacagctgcccagagcccccagcccctaccctagtctgatgacccaggcctttccccctgcccaggctagccttgtgctcagctgccagcacagcccccaccacacactccccaacaggtgtctccacaactgccaggagacagctcagcttagctgctcttcccccaacccccaggcccagcagaatcacgtctaggagagtgggccacatgcctcttggaaggcaggtggcacaagaggttcatcccaccctccagcattacccagccccacaggacccacctcctgctcccagctccagcctgaaaggagctagtcctctccagcccctttgaaaagaccagctggacattctccagggagattctgccctacaggattagggagtaccctcacctgg

SEQ ID NO: 14 - DNA, 740 bp, Macaca mulatta
  misc_feature (132)..(137): E box
agccaccgtcccagatacggctaaggcccccacagagtgtgcatcctaaggtggcccagaggtcctcagaaagtgctaagaggtcttcctcagaggaatagcaagggtaggtgcctctggcatgtctgagccagatgatgggagctatcatgagacttttccacctggggtgaggggcctagaaaccccccatggtcccttctccaccagatgccttacatcccccacccccccacacacacagcttggagactagaagcccctggaatgcggcccaccctctctccagttgttccccaccagcccagctgctggtccctaaccccacccacctgttgggtccttagcaccagctgcccagagcccccaacccctaccctagtctgatgacccaggccctttcccctgcccagactagccctgtgctcagctgccagcacagcccccaccacacactctccaacaggtgtctccacaactgccaggagacagctcagcttagctgctctttcctctacccccaggcccagcagaatcacatctaggagagtgggccacatgcctcttggaaggcaagtggcacatgtggttcatcccaccctccagcattacccagccccacagaacccacctcctgctcccagctccagcctgaaaggagctaggcctctccagcccctttgaaaagaccagctggccattctccagggagattttgccctacaggattagggagtacc

SEQ ID NO: 15 - DNA, 702 bp, Canis familiaris
  misc_feature (82)..(87): E box
aggagtccctgcagcgtgtgcacactggggccgcccaaaagtgctcagacagaggactttcttgggtggaaaagcctgtcacacctgggcttgatgctgggggccaccacaggatgtcaccactgtgcagaagggcccagagggacccccattgtgccctctcctgcccaaggcccccgtgtccacccccacatacacacacacacacacacacacacacacacacacacacacacagctcaggcactgtgacgtggccccaggtggcccactctgactccagttgttaccagtccagctgccaagcccctacggcccatctgtcaggccccaggtccccagtgtcagctgcccaaagtcctcctccccaggtccccccaagcctgcaggccctgctctcagctgccaccagggcccccaccacaccctcccccacaggcgtccgtcccgctgtccccaagccagcagagttgtgttgggggaggggggcagaccacttgtcacttcagaaggcaggtgacatgaggggtccagccaccagcagagtcacgcagcccctgcactagccctgtgggacccccacctcgctacgctcccccttccagcctggacagggtcaggcctctccgtccccttgcacagaccagcccgagctccccaaggagcttctgcccagcaggacccaggactgtcctctcctgg

SEQ ID NO: 16 - DNA, 743 bp, Equus caballus
  misc_feature (70)..(75): E box
aacactccctcagggtgcgcatcccagggccgcccagaagtcctcagaaaagcaaaaagaggcctttctcagatggaaaaggctgtggtgcatctgagccaaatgctgggggctgtcacaggacggtgccactgggggtgaggggtccagcaagtggccccattgtcctttcttttacaccagatcccctctgcccgcccccctcgcccccccgacacacacacacacagcttaggggtcggatctgccttctgggatgcagcccaccctgccgccagttgttgccagcccagctgctgagccccccaccccggcccacctgtcaggcccgcgggtcctcagcgccagctgcccagagcccccagccccagtctgatgacccaggcccttcccccggccaggccccgctctcagctgccagcacagcccccaccacaccctccccaacaggtgtccccacaactgcagggagatgccccggcagaattgtgccgggggggggggggggggggggctggggaggggacccctgtccccttcggaagggaggtgggaagaggggtccggcccagcctccgggacagggacccaagccgggccccagcctcggagaatttccacctccttctgctccaagttccagcctggactgggtcagggatctcctggccttatgaaaagaccaaccagaatccccccgggggaagttctgcccagcggggatcctggagagtcacgcctgg

SEQ ID NO: 17 - DNA, 6 bp, Artificial Sequence
  E-boxes
canntg

SEQ ID NO: 18 - DNA, 30 bp, Artificial Sequence
  forward primer
aagcggccgcacccagtttgcccagtgtag

SEQ ID NO: 19 - DNA, 28 bp, Artificial Sequence
  reverse primer
aacggccgccaggtaaagacaccccaga